(19) United States
(12) Patent Application Publication
(10) Pub. No.: US 2006/0149076 A1
(43) Pub. Date: Jul. 6, 2006

(54) Copper catalyzed arylation

(76) Inventor: Frederick Hicks, Somerville, MA (US)

Correspondence Address: KEVIN E. MC VEIGH;RHODIA INC., 259 PROSPECT PLAINS RD., CRANBURY, NJ, 08512, US

(21) Appl. No.: 10/629,463

(22) Filed: Jul. 29, 2003

Related U.S. Application Data
(60) Provisional application No. 60/400,662, filed on Aug. 2, 2002.

(51) Int. Cl. C07D 263/18; C07D 263/04; C07C 231/14

(52) U.S. Cl. 548/225; 548/316.4; 564/86; 564/123; 564/47

(57) ABSTRACT

The present invention is directed to an arylation of a nuclophile by reacting the nucleophile with a substrate aromatic compound ArX in the presence of a copper catalyst, a base and water, wherein Ar is aryl, heteroaryl or alkenyl, X is halo, sulfonate or phosphonate, the base comprises an alkaline earth carbonate, bicarbonate, hydroxide or phosphate, and the copper catalyst comprises a copper atom or ion and a ligand.

### FIELD OF THE INVENTION

This invention relates to aromatic bond formation, more specifically to copper-catalyzed formation of aryl and heteroaryl carbon-nitrogen bonds.

### BACKGROUND OF THE INVENTION

Palladium catalyzed arylation of indoles is known, see, for example, Stephen L. Buchwald Org. Lett., 2, 1403-1406. More recently, copper catalyzed arylation of indoles and amides has been reported, see, for example, Klapars, A., Antilla, J. C., Huang, X., and Buchwald, S. L., J. AM. Chem. Soc. 2001, 123, 7727-7729 and Buchwald, S. L. J. Am. Chem. Soc. 2002, 124, 7421. Buchwald et. al. disclose copper-catalyzed indole arylation with catalytic amounts, that is, from 0.1 to 10 mol %, copper under mild temperatures in solvents such as toluene and 1,4-dioxane and inorganic carbonate or phosphate bases such as K₂CO₃, Cs₂CO₃, or K₃PO₄. The key to this reactivity under mild conditions even in noncoordinating solvents are the use of ligands, for example, diamines, such as 1,2-diaminocyclohexane or N,N′-dimethylethylenediamine.

Current methods for copper-catalyzed indole arylation employing aryl bromides suffers from slow reaction rates, a variable induction period, use of large amounts of inorganic bases such as K₃PO₄, which lead to reactor agitation issues at minimal solvent volumes or without organic solvents and reactor capacity issues as well.

### SUMMARY OF THE INVENTION

The present invention is directed to cross-coupling reaction method for arylating a nuclophile comprising reacting the nucleophile with a substrate aromatic compound ArX in the presence of a copper catalyst, a base and water, wherein Ar is aryl, heteroaryl or alkenyl, X is halo, sulfonate or phosphonate, the base comprises an alkaline earth carbonate, bicarbonate, hydroxide or phosphate, and the copper catalyst comprises a copper atom or ion and a ligand.

The method of the present invention allows the amount of base to be reduced compared to prior methods, thus minimizing reactor agitation and capacity issues.

In one embodiment, the present invention is directed to a method for arylating a HN-containing heterocycle, comprising reacting the HN-containing heterocycle with a substrate aromatic compound ArX according to the reaction scheme: 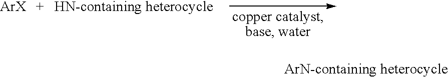
wherein Ar is aryl, heteroaryl or alkenyl,
X is halo, sulfonate or phosphonate,
the base comprises an alkaline earth carbonate, bicarbonate, hydroxide or phosphate, and
the copper catalyst comprises a copper atom or ion and a ligand.

In a second embodiment, the present invention is directed to a method for arylating a HN-containing compound according to the formula HN(R¹)R², comprising reacting the HN-containing compound with a substrate aromatic compound, ArX, according to the reaction scheme: 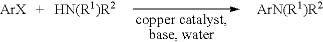
wherein Ar is aryl, heteroaryl or alkenyl,
X is halo, sulfonate or phosphonate
R¹ is is H, alkyl or aryl
R² is according to the formula: 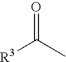
wherein R³ is H, alkyl, aryl, heteroaryl, alkenyl, —OR⁵ or —NR⁶₂, and R⁵ and R⁶ are each independently alkyl, aryl, or 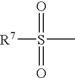
wherein R⁷ is alkyl or aryl,
the base comprises an alkaline earth carbonate, bicarbonate, hydroxide or phosphate, and
the copper catalyst comprises a copper atom or ion and a ligand.

### DETAILED DESCRIPTION OF INVENTION AND PREFERRED EMBODIMENTS

As used herein, the term “substituted” denotes the conceptual replacement of a hydrogen atom of a given organic moiety with a substituent group other than a hydrogen atom and includes all permissible substituent groups, including acyclic hydrocarbon groups, alicyclic hydrocarbon groups, monocyclic aromatic hydrocarbon groups, polycyclic aromatic hydrocarbon groups, heteroacyclic groups, heterocyclic groups, fused ring systems and bridged ring systems, of which the substituents specifically described below are illustrative examples.

“Alkyl” refers to a linear, branched or cyclic saturated hydrocarbon group, preferably a (C₁-C₃₀) linear, branched or cyclic saturated hydrocarbon group that may, optionally, contain one or more heteroatoms, such as, for example, methyl, ethyl, propyl, n-butyl, isobutyl, t-butyl, neopentyl, cyclopentyl, hexyl, cyclohexyl, decyl, stearyl, eicosyl, methoxy, triacontyl, 2,5,7-trioxanonanyl, 2,5,8-triazadecenyl, and that may, optionally, be substituted at one or more positions with other moieties, such as, for example, alkyl, alkenyl, alkynyl, aryl, heterocyclyl, halo, hydroxy, sulfhydryl, hydroperoxy, carbonyl-containing groups (including, for example, carboxy, ketone, ester and aldehyde groups), alkyloxy, alkyldioxy, amino, amido, imino, hydrazino, nitro, cyanato, thiocyanato, mercapto, thiocarbonyl-containing groups (including, for example, thioketone groups, thioester groups and thioaldehyde groups), sulfonyl-containing groups (including, for example, sulfate, sulfonate and sulfamoyl groups), silyl, siloxy and phosphorus-containing substituent groups (including, for example, phosphoranyl, phosphinyl, phosphinothioyl, phosphinimyl). Such substituent groups may themselves be further substituted with, for example, any of the groups described above as suitable substituents for alkyl groups, to form compound substituent groups, such as, for example, aralkyl, aminoalkyl, haloalkyl, heterocyclylalkyl.

As used herein the term “heteroatom” means an element other than carbon, such as for example, oxygen, nitrogen and sulfur.

As used herein, “halo” means fluoro, chloro, bromo or iodo, “hydroxy” means —OH, “sulfhydryl” means —SH, “hydroperoxy” means —OOH, “carbonyl” means —C(O)—, “carboxy” means —COOH, a ketone group is a group containing a carbonyl moiety that is attached to two carbon atoms, an ester group is a group containing a —C(O)OR moiety, an aldehyde group is a group containing a —CHO moiety, “alkyloxy” means —OR′, “alkyldioxy” means —OOR′, “amino” is conceptually a derivative of NH₃ in which one or more hydrogen atoms are replaced by nonacyl organic groups and includes primary, secondary and tertiary amines, “amido” includes, for example, —C(O)NR₁₂, “imino” means ═NH, “hydrazino” includes, for example, —HNNR″₂, “nitro” is —NO₂, “cyanato” is —OCN, “thiocyanato” is —SCN, “mercapto” is —SH, “thiocarbonyl” is —C(S)—, a “thioketone group” is a group containing a thiocarbonyl moiety that is attached to two carbon atoms, a “thioester group” is —C(S)-, a “thioaldehyde” group is a group containing a —CHS moiety, “sulfonyl” is —SO2—, “sulfate” includes, for example, —OSO₂OR″, “sulfonate” includes, for example, —O₂SOR″, “sulfamoyl” includes, for example, —O₂SNR₁₂, “silyl” is —SiR₁₃, “siloxy” is —OSiR″₃, “phosphino” includes —PR″₂, “phosphoranyl” includes, for example, —PR″₄, “phosphinyl” includes, for example, —P(O)R″₂, “phosphinothioyl” includes, for example, —P(S)R″₂, “phosphinimyl” includes, for example, —P(NH)R″₂, “aralkyl” means an alkyl group substituted with an aryl group, such as, for example, benzyl, “aminoalkyl” means an alkyl group substituted with an amino group, such as, for example, dimethylaminoethyl, “haloalkyl” means an alkyl group substituted with a halogen atom, such as, for example, chloromethyl, “heterocyclylalkyl” means an alkyl group substituted with a heterocyclyl group, such as, for example, pyrrolidinylethyl, wherein, as used above in this paragraph, R is an organic group, R′ is alkyl and R″ is H, alkyl or aryl.

“Alkenyl” refers to a linear, branched or cyclic hydrocarbon group, preferably a (C₂-C₂₀) linear, branched or cyclic hydrocarbon group, that contains one or more carbon-carbon double bonds per group and that may, optionally, contain one or more heteroatoms, such as, for example, ethenyl, propenyl, allyl, isopropenyl, ethenylidenyl, cyclopentyl, cyclohexadienyl, azanonenyl, and that may, optionally, be substituted at one or more positions with other moieties, such as, for example, any of the possible substituents described above in respect to alkyl groups.

“Alkynyl” refers to a linear, branched or cyclic unsaturated hydrocarbon group, preferably a (C₂-C₂₀) unsaturated hydrocarbon group, that contains one or more carbon-carbon triple bonds per group and that may, optionally, contain one or more heteroatoms, such as, for example, ethynyl, propynyl, thianonynyl, and that may, optionally, be substituted at one or more positions with other moieties, such as, for example, any of the possible substituents described above in respect to alkyl groups.

“Aryl” refers to an unsaturated hydrocarbon group that contains one or more six membered rings in each of which the unsaturation may be represented by three conjugated carbon-carbon double bonds, including monocyclic and polycyclic ring systems, such as, for example, phenyl, naphthyl, anthryl, phenanthryl, indenyl, fluorenyl, which may, optionally, be substituted at one or more positions with other moieties, such as, for example, any of the possible substituents described above in respect to alkyl groups.

“Heterocyclyl” and “heterocycle” refer to a saturated or unsaturated organic group or compound that contains one or more rings in which one or more ring members is a heteroatom, preferably a nitrogen, sulfur or oxygen heteroatom, such as, for example, thiacyclopentadienyl, thiaindenyl, thianthrenyl, oxacyclopentadienyl, oxaindenyl, isobenzylfuranyl, pyranyl, azacyclopentadienyl, pyridinyl, pyrazinyl, pyrimidinyl, pyridazinyl, indolinyl, quinolinyl, isoquinolinyl, phthalazinyl, cinnolinyl, azafluorenyl, phenanthrolinyl, phenazinyl, phenothiazinyl, phenarsazinyl, isothiazolyl, isoxazolyl, phenoxazinyl, pyrrolidinyl, pyrimadinyl, imidazolidinyl, piperidinyl, piperizinyl, oxathiaanthracenyl, isoxazolyl, oxaazaanthracenyl, isothiazolyl, morpholinyl, and which may, optionally, be substituted at one or more positions with other moieties, such as, for example, any of the possible substituents described above in respect to alkyl groups.

As used herein, the term “nucleophile” refers to a chemical moiety having a reactive pair of electrons and the terms “electrophile” and “electrophilic” refer to a chemical moiety that can accept a pair of electrons from a nucleophile.

Compounds suitable as the substrate aromatic compound ArX component of the method of the present invention are those compounds that contain an electrophilic atom bonded to leaving group X that is susceptible to the above reaction with a nucleophile.

In one embodiment, Ar comprises a phenyl ring, which may in addition to the X substituent, be further substituted on one or more carbons of the ring with, for example, any of the groups described above as suitable substituents for alkyl groups. In a preferred embodiment, Ar comprises a phenyl ring, which is further substituted, in addition to the X substituent, on one or more carbons of the ring with one or more substituent groups each independently selected from alkyl, alkoxy, alkenyl, alkynyl, aryl, heteroaryl, cyano, carbonyl, amino, amido or sulfonyl.

In one embodiment, X is a halo, sulfonate or phosphonate group, more preferably halo.

Suitable sulfonate groups include, for example, those according to the general formula:

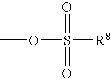

wherein R⁸ is alkyl, aryl, fluoroalkyl, preferably trifluoromethyl, perfluoroalkyl.

Suitable phosphonate groups include, for example, those according to the general formula: 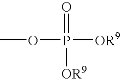
wherein each R⁹ is independently alkyl or aryl.

Compounds suitable as the substrate aromatic compound ArX include, for example, 4-bromobenzonitrile, 4-N,N′-dimethyl-bromoaniline, 2-bromothiophene, 3-bromoquinoline, 1-nitro-2-iodobenzene, 4-chlorotoluene, 4-bromofluorobenzene, 2-bromoanisole, 4-iodoaniline, 3-bromoacetophenone, and 4-bromothioanisole. In a highly preferred embodiment, the substrate aromatic compound is 4-bromofluorobenzene.

Suitable substrate aromatic compounds are made by known synthetic methods. The substituents on the substrate aromatic compound are selected based on structure of the desired product.

Compounds suitable as the nucleophile component of the method of the present invention include HN-containing heterocycles and HN-containing compound according to the formula HN(R¹)R², wherein R¹ and R² are as described above.

Compounds suitable as the HN-containing heterocycle component of one of the preferred embodiments of the method of the present invention include, for example, substituted or unsubstituted triazoles, pyrroles, pyrazoles, imidazoles, indoles, azaindoles, benzotriazoles, benzimidazoles, indazoles, and carbazoles, such as, for example, 3-methylpyrazole, 2-phenylindole, 5-methoxyindole, 5-aminoindole, 5-nitroindole, 3-carbomethoxyindole, benzimidazolylacetonitrile, pyrrole, 7-azaindole, 1,2,4-triazole, and carbazole.

In one embodiment, the HN-containing heterocycle comprises a monocyclic system according to the formula: 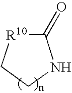
wherein n is 0 or an integer of from 1 to 3 and R¹⁰ is substituted alkyl, substituted N, or O, such as, for example, 2-pyrimidinone, phthalazinone, 2-azetidinone, 2-pyrrolidinone, 2-oxazolidinone, or imidazolidinone.

In a preferred embodiment, the N-containing heterocycle is 5-chloroindole or 2-pyrrolidinone.

Compounds suitable as the HN-containing compound according to the formula HN(R¹)R² include amides, carbamates, ureas, and sulfonamides.

In one embodiment, the HN-containing compound according to the formula HN(R¹)R² is an amide, such as for example, benzamide, 4-aminobenzamide, cyclohexylamide, trans-cinnamamide, N-phenylacetamide, N-methylformamide, N-benzylformamide, or N-cyclohexylformamide. In a more highly preferred embodiment, the HN(R¹)R² compound is selected from benzamide and N-methylformamide.

In a another embodiment, the HN-containing compound according to the formula HN(R¹)R² is a carbamate, urea or sulfonamide, such as, for example, N-phenyl-tert-butyl carbamate, N-methylimidazolidinone, or p-toluenesulfonamide.

The copper atom or ion component of the method of the present invention may be derived from any copper-containing material. In a preferred embodiment, the copper atom or ion is derived from copper metal, Cu₂O or a copper salt, such as CuCl, CuBr, CuBr₂ or CuI. In a preferred embodiment, the copper catalyst is CuI.

In a preferred embodiment, the copper catalyst is present in the reaction mixture as a metal-ligand complex wherein the copper catalyst is bound to a supporting ligand.

Compounds suitable as the ligand component of the method of the present invention are those compounds that are capable of solubilizing the copper species in the reaction mixture. In a preferred embodiment, the ligand is a 1,2-diamine compound, such as, for example, 1,2-di(aminomethyl)cyclohexane, N,N′-dimethylethylenediamine, 1-propyl-1,2-N,N′-dimethylethylenediamine. In a more highly preferred embodiment, the ligand is 1,2-di(aminomethyl)cyclohexane.

The copper atom or ion component and ligand component may be added to the reaction mixture as separate compounds. Alternatively, a copper-ligand catalyst complex may be formed prior to addition to the reaction mixture and then added to the reaction mixture as the copper-ligand complex.

In general, the coupling reaction is run in the presence of a catalytic amount of the copper catalyst. As used herein, a “catalytic amount” of catalyst refers to an amount of catalyst that provides an increase in the rate of the reaction of the method of the present invention, compared to the rate of the same reaction conducted under analogous conditions, but absent the catalyst. Typically, the amount of copper catalyst ranges from about 0.01 to about 10 mole %, more preferably from about 0.5 to about 5 mole %, based on the amount of limiting reactant.

In a preferred embodiment, the coupling reaction is run in the presence of from about 0.8 to 3 equivalents, more preferably from about 1.0 to about 2.0 equivalents of ArX, based on the amount of nucleophile.

Compounds suitable as the base component of the method of the present invention include, for example, magnesium bicarbonate, potassium carbonate, cesium carbonate, potassium phosphate, sodium hydroxide and potassium hydroxide. In a preferred embodiment, the base comprises one or more of sodium hydroxide and potassium hydroxide. In a preferred embodiment, the reaction mixture includes from about 1 to about 5 equivalents, more preferably from about 1.2 to about 4 equivalents, of base, based on the amount of nucleophile.

The base is generally added to the reaction mixture as solid or as an aqueous solution. The full amount of base to be used in the reaction may be added to the reaction mixture at one time or may be added to the reaction mixture over time. In any case, it is preferred that the base be added after formation of the copper-ligand catalyst complex.

In a preferred embodiment, the reaction is conducted in the presence of from about 1 to about 80 percent by volume (“vol %”) water, more preferably from about 10 to about 50 vol % water, based on the total volume of reaction mixture.

In one embodiment, the reaction mixture contains no organic solvent and in a preferred embodiment, the reaction mixture consists essentially of the reactants, copper catalyst, base and water.

In an alternative embodiment, the reaction mixture further comprises a solvent selected from aliphatic or aromatic hydrocarbon solvents such as pentane, hexane, benzene, xylene and toluene, ethers such as diethyl ether and t-butyl methyl ether, tetrahydrofuran, 1,4-dioxane and 1,2-dimethoxyethane.

In general, the coupling reaction is run under mild conditions that will not adversely affect the reactants, catalyst or product. In a preferred embodiment, the coupling reaction is run at a temperature of from about 25° C. to about 300° C., more preferably from about 25° C. to about 150° C.

In a preferred embodiment, the coupling reaction is run in an inert atmosphere, such as, for example, under an argon or nitrogen atmosphere.

### Examples 1 and 2 and Comparative Examples C1 and C2

### Synthesis of 5-Chloro-1-(4-Fluorophenyl)Indole

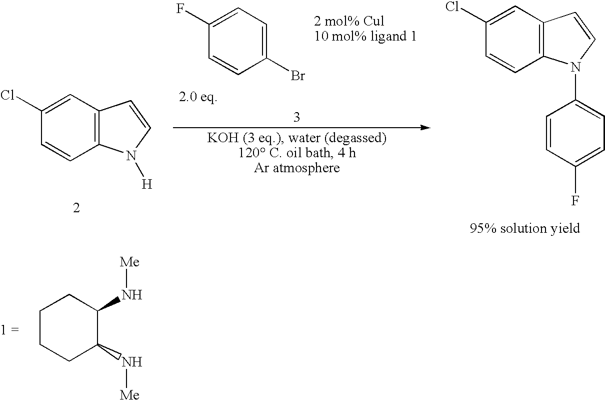

In the reaction of Example 1, a reaction vessel composed of a 250 mL 3-necked round bottom flask equipped with a reflux condenser, overhead mechanical stirrer and Ar inlet was flushed with Ar for 30 mins. The reaction vessel was then charged with 4-bromofluorobenzene 3 (6.6 mL, 60 mmol) and H₂O (5 mL, purged with bubbling Ar for 1 h) and the overhead stirrer was engaged. 1,2-di(aminomethyl)cyclohexane 1 (426 mg, 3 mmol), CuI (114 mg, 0.60 mmol), and 5-chloroindole 2 (4.5 g, 30 mmol) were added and reaction mixture was stirred for 5 minutes. KOH (5.05 g, 90 mmol) was charged to the reaction vessel and the reaction flask was placed in a 120° C. oil bath. After 4 hours, 95% conversion was indicated by GC analysis of aliquots diluted with EtOAc. The oil bath was removed and hot reaction mixture was charged with H₂O (4 volumes), which led to a rapid precipitation of the organic product. Once reaction mixture had cooled to ambient temperature, the solid product was collected by filtration and dried overnight on house vacuum. Yield: 7.03 g (95%). GC Purity: 97.5%.

The reactions of Example 2 and Comparative Examples C1 and C2 were conducted under conditions analogous to those of Example 1, except as indicated in TABLE I:

TABLE I

| Example # | Water | Base | |
|---|---|---|---|
| Ex. 1 | 5 mL | 5.05 | g KOH |
| Ex. 2 | 5 mL | 14.88 | g K₃PO₄ |
| C. Ex. C1 | none | 5.05 | g KOH |
| C. Ex. C2 | none | 14.88 | g K₃PO₄ |

The percent conversion over time is given below in TABLE II for each of the examples.

TABLE II

| | Conversion (%) | | |
|---|---|---|---|
| Example # | 1 hour | 2 hours | 4 hours |
| Ex. 1 | 56 | 86 | 96 |
| Ex. 2 | 42 | 66 | 90 |
| C. Ex. C1 | 2 | 3.7 | 6 |
| C. Ex. C2 | 54 | 65 | 83 |

The use of water/KOH offered comparable kinetics to using 2 eq ArBr as solvent with 2 eq K₃PO₄, either with or without water, and provided the advantages of 1) a cheaper and more volume efficient base and 2) easier reaction agitation which should help scale up.

1. A method for arylating a nuclophile, comprising reacting the nucleophile with a substrate aromatic compound, ArX, in the presence of a copper catalyst, a base and water, wherein Ar is aryl, heteroaryl or alkenyl, X is halo, sulfonate or phosphonate, the base comprises an alkaline earth carbonate, bicarbonate, hydroxide or phosphate, and the copper catalyst comprises a copper atom or ion and a ligand.

2. The method of claim 1, wherein the nucleophile comprises a HN-containing heterocycle or a HN-containing compound according to the formula HN(R¹)R², wherein R¹ is H, alkyl or aryl, and R² is a substituent according to the formula: 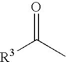
wherein R³ is H, alkyl, aryl, heteroaryl, alkenyl, —OR⁵ or —NR⁶₂, and
R⁵ and R⁶ are each independently alkyl, aryl, or a substituent according to the formula: 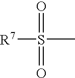
wherein R⁷ is alkyl or aryl.

3. The method of claim 2, wherein the nucleophile comprises a HN-containing heterocycle selected from triazoles, pyrroles, pyrazoles, imidazoles, indoles, azaindoles, benzotriazoles, benzimidazoles, indazoles, and carbazoles, each of which may be substituted or unsubstituted.

4. The method of claim 2, wherein the nucleophile comprises a HN-containing heterocycle comprises a monocyclic system according to the formula: 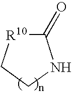
wherein n is 0 or an integer of from 1 to 3 and R¹⁰ is substituted alkyl, substituted N, or 0.

5. The method of claim 4, wherein nucleophile comprises an amide, a carbamate, a urea, or a sulfonamide.

6. The method of claim 5, wherein nucleophile comprises benzamide, 4-aminobenzamide, cyclohexylamide, trans-cinnamamide, N-phenylacetamide, N-methylformamide, N-benzylformamide, N-cyclohexylformamide, N-phenyl-tert-butyl carbamate, N-methylimidazolidinone, or p-toluenesulfonamide.

7. The method of claim 1, wherein Ar comprises a phenyl ring which is, other than the X substituent, unsubstituted or is further substituted, in addition to the X substituent, on one or more carbons of the ring with one or more substituent groups each independently selected from alkyl, alkoxy, alkenyl, alkynyl, aryl, heteroaryl, cyano, carbonyl, amino, amido or sulfonyl.

8. The method of claim 1, wherein X is a halo.

9. The method of claim 1, wherein X is a sulfonate group according to the formula: 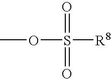
wherein R⁸ is alkyl, aryl, fluoroalkyl, preferably trifluoromethyl, perfluoroalkyl.

10. The method of claim 1, wherein X is a phosphonate group according to the formula: 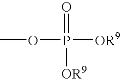
wherein each r⁹ is independently alkyl or aryl.

11. The method of claim 1, wherein ArX comprises 4-bromobenzonitrile, 4-N,N′-dimethyl-bromoaniline, 2-bromothiophene, 3-bromoquinoline, 1-nitro-2-iodobenzene, 4-chlorotoluene, 4-bromofluorobenzene, 2-bromoanisole, 4-iodoaniline, 3-bromoacetophenone, or 4-bromothioanisole.

12. The method of claim 1, wherein the copper atom or ion is derived from copper metal, from Cu₂O or from a copper salt selected from CuCl, CuBr, CuBr₂ and CuI.

13. The method of claim 1, wherein the ligand comprises a 1,2-diamine compound.

14. The method of claim 1, wherein the ligand comprises 1,2-di(aminomethyl)cyclohexane, N,N′-dimethylethylenediamine, or 1-propyl-1,2-N,N′-dimethylethylenediamine.

15. The method of claim 1, wherein the copper catalyst is present in the reaction mixture as complex comprising the copper atom or ion and the ligand.

16. The method of claim 1, wherein the copper catalyst is present in an amount of from about 0.01 to about 10 mole %, based on the amount of limiting reactant.

17. The method of claim 1, wherein the reaction is conducted in the presence of from about 0.8 to 3 equivalents of ArX, based on the amount of nucleophile.

18. The method of claim 1, wherein the reaction is conducted in the presence of from about 1 to about 80 percent by volume water, based on the total volume of reaction mixture.

19. A method for arylating a HN-containing heterocycle, comprising reacting the HN-containing heterocycle with a substrate aromatic compound, ArX, according to the reaction scheme: 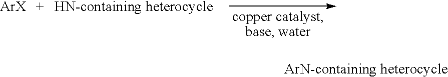
wherein Ar is aryl, heteroaryl or alkenyl,
X is halo, sulfonate or phosphonate,
the base comprises an alkaline earth carbonate, bicarbonate, hydroxide or phosphate, and
the copper catalyst comprises a copper atom or ion and a ligand.

20. A method for arylating a HN-containing compound according to the formula HN(R¹)R², comprising reacting the HN-containing compound with a substrate aromatic compound, ArX, according to the reaction scheme: 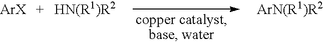
wherein Ar is aryl, heteroaryl or alkenyl,
X is halo, sulfonate or phosphonate
R¹ is H, alkyl or aryl
R² is according to the formula: 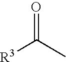
wherein R³ is H, alkyl, aryl, heteroaryl, alkenyl, —OR⁵ or —NR⁶₂, and R⁵ and R⁶ are each independently alkyl, aryl, or 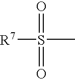
wherein R⁷ is alkyl or aryl,
the base comprises an alkaline earth carbonate, bicarbonate, hydroxide or phosphate, and
the copper catalyst comprises a copper atom or ion and a ligand.
